(19) United States
(12) Patent Application Publication
(10) Pub. No.: US 2023/0346775 A1
(43) Pub. Date: Nov. 2, 2023

(54) CANCER TREATMENT WITH TLR AGONIST

(71) Applicants: Lixin LI, Edison, NJ (US); Robert ANDTBACKA, Edison, NJ (US); Tao HUANG, Edison, NJ (US); BIRDIE BIOPHARMACEUTICALS, INC., Grand Cayman (KY)

(72) Inventors: Lixin Li, Edison, NJ (US); Robert Andtbacka, Edison, NJ (US); Tao Huang, Edison, NJ (US)

(21) Appl. No.: 18/023,306

(22) PCT Filed: Aug. 26, 2021
(86) PCT No.: PCT/US2021/047826

§ 371 Date: Feb. 24, 2023

Related U.S. Application Data
(60) Provisional application No. 63/070,376, filed on Aug. 26, 2020.

(51) Int. Cl. A61K 31/4745; A61K 39/395; A61K 9/00; A61K 47/12; A61P 35/00; G01N 33/574

(52) U.S. Cl. CPC A61K 31/4745; A61K 39/3955; A61K 9/0019; A61K 47/12; A61P 35/00; G01N 33/57488

(57) ABSTRACT

The present specification provides methods of monotherapy for cancer using an imidazoquinolinamine drug. Further methods provide for coordination with immune checkpoint inhibitor therapy for cancer.

### CROSS REFERENCE TO RELATED APPLICATIONS

The present application is a national stage entry under 35 USC § 371 of international patent application no. PCT/US21/47826 filed Aug. 26, 2021, which claims priority of U.S. provisional patent application 63/070,376 filed Aug. 26, 2020, the entire content of each of which is incorporated by reference herein in its entirety.

### BACKGROUND

The therapeutic potential of imidazoquinolinamines, such as imiquimod and Compound A, has long been recognized. Realizing that potential has proved challenging. The imidazoquinolinamines are typically agonists of toll-like receptor (TLR) 7 and/or TLR8 and have both antiviral and anticancer activity in vitro. Imiquimod has been successfully developed into a topical treatment for actinic keratosis and genital and perianal warts. However, no imidazoquinolinamine has been successfully developed as a systemically administered drug, due to dose limiting toxicity (DLT).

### SUMMARY

Disclosed herein are dosages and patient populations in which an imidazoquinolinamine drug, 1-[4-amino-2-(ethoxymethyl)imidazo[4,5-c]quinolin-1-yl]-2-methylpropan-2-ol, or a pharmaceutically aceptable salt thereof, collectively Compound A (unless context dictates otherwise), can be administered systemically as monotherapy for the treatment of cancer with therapeutic benefit and acceptable toxicity. In some embodiments, Compound A is administered in the form of the free base or conjugate base. In some embodiments, Compound A is administered in the form of a pharmaceutically acceptable salt. Further embodiments include formulations thereof. Dosages described herein are based on the free base, even when a salt, such as the sulfate salt, are being administered (unless otherwise noted). In some embodiments, dosages are adjusted based on the molecular weight of the particular species being administered.

Some embodiments are methods of treating cancer in a patient in need thereof comprising administering Compound A at a dosage of from 0.10 to 1.2 mg/m². In some embodiments, the dosage is at least 0.10, 0.15, 0.30, 0.45, or 0.6 mg/m². Some embodiments comprise administering Compound A at a dosage not exceeding 0.75, 0.9, 1.0, 1.1, or 1.2 mg/m². In some embodiments, the administered dosage of Compound A is in a range bounded by any of these values. In some embodiments, the patient is human.

In some embodiments, Compound A is used as monotherapy; that is, treatment with Compound A does not commence until after the last dose of any prior cancer therapy and no other anti-cancer drug is administered in the same time frame in which Compound A is being administered. In some embodiments, treatment with Compound A does not commence until concentrations of the prior anti-cancer drug in the patient's body have substantially subsided or have been eliminated. In some embodiments, treatment with Compound A does not commence until at least 2 weeks or at least 4 weeks after the last administration of the prior anti-cancer drug. In some embodiments, treatment with Compound A commences within 3 month or 6 months of the last administration of the prior anti-cancer drug. In some embodiments, treatment with Compound A does not commence until after the point in time when a next dose of prior therapy would have been given had such treatment not been discontinued.

In some embodiments, Compound A is more effective after another cancer therapy (even if the patient's cancer progressed on the other therapy). In some embodiments, the prior therapy was chemotherapy, for example cytotoxic therapy or targeted therapy. In some embodiments, the prior therapy was immune checkpoint inhibition therapy, for example PD-1 blockade.

Some embodiments are methods of treating cancer in a patient in need thereof comprising administering Compound A after a course of treatment of immune checkpoint inhibitor therapy comprising at least 1, 2, 3, 4, 5, or 6 doses of the immune checkpoint inhibitor (or any range of doses bound by those values). In some embodiments, administration of the immune checkpoint inhibitor is discontinued at about, or prior to, the time treatment with Compound A is initiated. That is, Compound A is administered after a last dose of the immune checkpoint inhibitor, but not more than 3 months afterward. In some embodiments, treatment with Compound A is initiated after the initial doses of the immune checkpoint inhibitor have been administered, but the immune checkpoint inhibitor therapy is not discontinued. In some embodiments, the patient's cancer progresses during the initial immune checkpoint inhibitor therapy, and treatment with Compound A is initiation after observation of that progression. In some embodiments, the immune checkpoint inhibitor therapy is PD-1 blockade.

In some embodiments, PD-1 blockade comprises administration of an anti-PD-1 antibody. In other embodiments, PD-1 blockade comprises administration of an anti-PD-L1 antibody.

Some embodiments are methods of treating cancer comprising administering an therapeutically effective amount of Compound A, or a pharmaceutically acceptable salt thereof, to the patient to provide a plasma concentration profile in said patient comprsing:
a. a maximum plasma concentration (Cₘₐₓ) of Compound A free base of at least 8 ng/mL; or
b. an area under the curve (AUC) of Compound A free base of at least 3 ng/mL*Day; or
c. both;
   whereby the likelihood of clinical benefit is increased as compared to treatment with Compound A that does not attain the plasma concentration profile.

Some embodiments are methods of treating cancer comprising administering an therapeutically effective amount of Compound A, or a pharmaceutically acceptable salt thereof, and an anti-PD-1 antibody to the patient to provide a plasma concentration profile in said patient comprsing:
a. a maximum plasma concentration (Cₘₐₓ) of Compound A free base of at least 7 ng/mL; or
b. an area under the curve (AUC) of Compound A free base of at least 2 ng/mL*Day; or
c. both;
   whereby the likelihood of clinical benefit is increased as compared to treatment with Compound A that does not attain the plasma concentration profile.

Some embodiments are methods of treating cancer comprising administering an therapeutically effective amount of Compound A, or a pharmaceutically acceptable salt thereof, and an anti-PD-L1 antibody to the patient to provide a plasma concentration profile in said patient comprsing:
a. a maximum plasma concentration (Cₘₐₓ) of Compound A free base of at least 10 ng/mL; or
b. an area under the curve (AUC) of Compound A free base of at least 2 ng/mL*Day; or
c. both;
   whereby the likelihood of clinical benefit is increased as compared to treatment with Compound A that does not attain the plasma concentration profile.

In some embodiments, Compound A is administered parenterally. In some embodiments, parenteral administration comprises intravenous injection or infusion. In some embodiments, Compound A is infused over 15-90 minutes, or over 30-60 minutes.

Some embodiments further comprise determining a change in interferon inducible protein 10 (IP-10) plasma or whole blood concentration from before to after Compound A administration. Some embodiments further comprise determining a change in IP-10 transcripts in plasma or whole blood from before to after Compound A administration. In some embodiments, IP-10 concentration or transcript level after Compound A administration is determined after 3 or 6 administrations of Compound A. In various aspects of these embodiments, the IP-10 is increased greater than 2-, 3-, 4-, or 5-fold. In some embodiments, the dosage of Compound A is increased if IP-10 concentration in plasma or whole blood has not increased by a threshold level, for example, greater than 2-, 3-, 4-, or 5-fold.

In some embodiments, Compound A is administered at a dosage sufficient to produce a Cmax of Compound A free base in plasma that is at least 9.4 ng/ml in the patient. In some embodiments, Compound A is administered at a dosage sufficient to produce an AUC of Compound A free base in plasma that is at least 2.09 ng/mL*day in the patient. If one or the other of these thresholds is not met, dosage of Compound A may be increased. In some embodiments, Tmax is from 15 to 90 minutes after administration. In some embodiments, Tmax is within 60 minutes after administration. In some aspects, after administration refers to after beginning infusion.

In some embodiments, when increasing Compound A dosage, it is increased by 0.15, 01, or 0.05 mg/m².

Compound A can be administered by 20-90 minute intravenous infusion, 1-6 times over a period of 3 to 6 weeks, with administration not occurring more often than once per week. In a particular embodiment, Compound A is administered weekly in a 3-week cycle, that is, on days 1, 8, and 15 of a 21 day cycle. In another particular embodiment, the Compound A is administered once in a 3-week cycle, that is, on day 1 of a 21-day cycle. In another particular embodiment, Compound A is administered once in a 6-week cycle, that is, on day 1 of a 42 day cycle. Further particular embodiments consistent with the above stated pattern are also contemplated. These embodiments are freely combinable with other variable aspects of the herein disclosed methods of treatment, including dosage, dose adjustment, and patient population, such as according to cancer type or prior therapy.

### BRIEF DESCRIPTION OF DRAWINGS

FIG. 1 depicts the pharmacokinetics of salt form of Compound A in escalating doses, by measuring the concentration of free base form of Compound A in plasma over time at Cycle 1 Day 1 post first infusion. Each circle indicates average plasma concentration of Compound A free base detected by mass spectrometry. Quantification of Compound A in plasma samples was analyzed for subjects in Dose Levels 1 (0.30 mg/m²; n=8), 2 (0.45 mg/m²; n=8), 3 (0.60 mg/m²; n=7), and 4 (0.75 mg/m²; n=11).

FIGS. 2A-B present analyses of interferon inducible protein 10 (IP-10) production in response to various doses of Compound A. FIG. 2A depicts the induction from baseline IP-10 production in log scale in response to four dose levels of Compound A (1-0.30 mg/m²; 2-0.45 mg/m²; 3-0.60 mg/m²; 4-0.75 mg/m²). The parenthetical numeral following the Dose Level indicator is the number of subjects. The interquartile range is indicated by the exterior horizontal lines in each plot connected by a vertical line. Rectangle: interquartile range; Braces: min/max; Horizontal line: unity (no change from baseline. The interior horizontal line indicates the median, the lower line and the geometric mean, the upper line. * means p<0.05 (Wilcoxon Rank-Sum Test in log domain). FIG. 2B depicts a comparison of fraction of baseline induction of IP-10 production for various Compound A dose levels (D1-D4, corresponding to 1-4 in FIG. 1). Calculations performed in log domain (Wilcoxon Rank-Sum Test). Each horizontal line represents the 95% confidence interval. The solid black circle is the estimate of fold-change in IP-10 production. P-value appears to the right of each horizontal line. The dashed vertical line is the null value.

FIGS. 3A-B depict pharmacodynamic-pharmacokinetic (PD-PK) analysis of IP-10 changes versus plasma concentration of Compound A. FIG. 3A depicts fraction of baseline IP-10 induction at Cycle 1 Day 1 and Day 8 (open circles with connecting lines for each subject) for PK of Compound A by Cmax (left panel) and AUEC quartiles (right panel). The parenthetical numeral indicated above is the number of subjects at Cycle 1 Day 1 and Day 8 in each quartile. Median (thick black line) and geometric mean (square) are displayed within the interquartile range (grey braces). Grey horizontal line represents unity (no change in IP-10 induction from baseline) and the filled circles indicate fold of induction <1. FIG. 3B depicts IP-10 fraction of baseline induction from baseline AUEC compared between quartiles for Cmax (left panel) and AUC (right panel) quartiles. Each horizontal line represents the 95% confidence interval. The solid black circle is the estimate. P-value appears to the right of each horizontal line. The dashed vertical line is the null value. Calculations performed in log domain (Wilcoxon Rank-Sum Test).

FIG. 4 depicts IP-10 cytokine induction level of 32 patients post infusion on Cycle 1 were analyzed. IP-10 induction level was presented as fold induction from baseline and then divided into two groups in accordance with the pharmacokinetics parameter AUC for Compound A, 16 patients in a low AUC group (AUC below 2.09 ng/mL*Day) and 16 patients in a high AUC group (AUC above 2.09). IP-10 induction level of the two groups was calculated as mean +/−SEM and the statistical difference between two groups was analyzed by Student's T-test. The mean value of induction level of IP-10 from baseline is 3.68-fold and 27.2-fold for the low AUC and high AUC groups, respectively.

FIG. 5 depicts IP-10 cytokine induction level of 32 patients post infusion on Cycle 1 was analyzed. IP-10 induction level of was presented as fold induction from baseline and divided into two groups in accordance with the pharmacokinetics parameter Cmax for Compound A, 16 patients each in a low Cmax group (Cmax below 9.4 ng/mL) and a high Cmax group (Cmax above 9.4 ng/mL), respectively. IP-10 induction level of the two groups was calculated as mean +/−SEM and the statistical difference between two groups was analyzed by Student's T-test. The mean value of induction level is 5.09-fold and 31.9-fold for the low Cmax and high Cmax groups, respectively.

FIGS. 6A-C depict the percentage of patients achieving a partial response or long-term stable disease above and below a threshold for AUC and Cmax of Compound A free base in plasma upon treatment with Compound A monotherapy (FIG. 6A), combination therapy with Compound A and an anti-PD-1 mAb (FIG. 6B), and combination therapy with Compound A and an anti-PD-L1 mAb (FIG. 6C).

### DETAILED DESCRIPTION

Disclosed herein are methods of treating cancer with 1-[4-amino-2-(ethoxymethyl)imidazo[4,5-c]quinolin-1-yl]-2-methylpropan-2-ol,

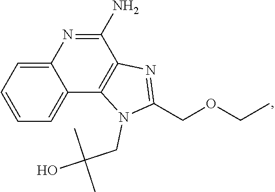 [SMILES: CCOCc1nc2c(N)nc3ccccc3c2n1CC(C)(C)O]

or a pharmaceutically aceptable salt thereof, collectively Compound A (unless context dictates otherwise). In some embodiments, Compound A is administered as monotherapy. In some embodiments, the patient has been previously treated with an immune checkpoint inhibitor, for example, a PD-1/L1 axis immune checkpoint inhibitor. In some embodiments, the immune checkpoint inhibitor therapy continues in conjunction with treatment with Compound A. In other embodiments, the immune checkpoint inhibitor therapy is discontinued prior to initiation of treatment with Compound A.

There has been much interest in, and research on, using Compound A for cancer therapy, but it has not been successfully put into clinical practice. The wide-spread perception is that Compound A has no therapeutic window in systemic use, and especially intravenous administration; that the toxic effects would overtake any therapeutic benefit. These issues are most acute in Compound A monotherapy. Nonetheless, herein disclosed are dosages and administration schedules for intravenous administration of Compound A, particularly pharmaceutically acceptable salts, as well as preferred patient populations, by which Compound A monotherapy can provide therapeutic benefit without causing an unacceptable level of toxicity. Without drug-drug interactions, this therapeutic benefit should persist in combination use with other therapeutics.

In some embodiments, the patient has been treated with some other cancer therapy prior to the initiation of treatment with Compound A. In various embodiments, the other therapeutic may be an immune checkpoint inhibitor, a chemotherapeutic such as a cytotoxic agent or a targeted therapeutic, or a therapeutic monoclonal antibody. In various embodiments, the therapeutic monoclonal antibody can be an antagonist or agonist immune checkpoint inhibitor, an anti-cancer antigen antibody, a don't-eat-me signal blocker, or an antibody mediating depletion of M2 macrophages. In some embodiments, the other treatment is discontinued prior to commencing treatment with Compound A, as described above. In other embodiments, the other treatment is continued in conjunction with treatment with Compound A. In still other embodiments, treatment with Compound A is suspended, the prior other treatment repeated and again discontinued, and treatment with Compound A resumed. Some embodiments specifically include one or more of the modes of treatment. Some embodiments specifically exclude one or more of the modes of treatment.

As used herein, monotherapy refers to a cancer treatment regimen utilizing a single anti-cancer therapeutic agent. It does not exclude prior (or subsequent) therapy with another therapeutic agent, but merely indicates that administration of the monotherapy agent is not coincident, or coordinated, in time with administration of another anti-cancer therapy. At a minimum, initiation of monotherapy does not commence until after the administration of a last dose of a prior agent. In some embodiments, treatment with Compound A does not commence until after the point in time when a next dose of prior anti-cancer therapy would have been given had such treatment not been discontinued. In some embodiments, initiation of the monotherapy does not commence until presence of a prior anti-cancer agent in the body has substantially subsided or been eliminated. In some embodiments, monotherapy is with respect to another anti-cancer pharmaceutical agent; a composition of matter administered to the patient's body. In such embodiments, the monotherapy may overlap, or be coordinated, in time with surgical treatment or radiation therapy.

As used herein, chemotherapy refers to the use of small molecule drugs for the treatment of cancer. Historically, chemotherapeutics have been drugs that damaged DNA or otherwise disrupted the mechanisms of cell division, and were cytotoxic to dividing cells generally. Such drugs can be referred to as cytotoxic or DNA-damaging chemotherapeutics. More recently, so-called targeted therapies have been developed. These also involve the use of small molecule drugs. However, these drugs act on specific proteins, very often kinases, involved in the regulation of cell division. These drug target proteins are generally expressed only in some cells of the body and may even be overexpressed in cancer cells. As a result, the effects of the drugs are more specific than the traditional cytotoxic chemotherapeutics. Some embodiments relate to patients who have received prior treatment with chemotherapy. In some of those embodiments, the chemotherapy was cytotoxic or DNA-damaging chemotherapy. In other of those embodiments the chemotherapy was targeted chemotherapy. Other embodiments specifically exclude prior treatment with any or all of these modes of chemotherapy.

Immune checkpoint inhibition therapy refers to the use of pharmaceuticals, typically biologics, that act on regulatory pathways in the differentiation and activation of T cells to promote the passage of T cell developmental program through these checkpoints so that anti-tumor (or other therapeutic) activity can be realized. The agents bringing about immune checkpoint therapy are commonly called immune checkpoint inhibitors and it should be understood that it is the check on T cell development that is being inhibited. Thus, while many immune checkpoint inhibitors also inhibit the interaction of receptor-ligand pairs (e.g., programmed cell death 1 (PD-1) interaction with programmed death-ligand 1 (PD-L1)), other checkpoint inhibitors (such as anti-OX40, anti GITR, anti-CD137, anti-CD122, anti-CD40, and anti-ICOS) act as agonists of targets that release or otherwise inhibit the check on T cell development, ultimately promoting effector function and/or inhibiting regulatory function. While inhibition of some checkpoints has proven to be sufficient to mediate clinical improvement in some instances, inhibition of other checkpoints works best in combinations. Most commonly, antibodies against one member of the receptor-ligand pair are used. In alternative embodiments the antibody is replaced with another protein that similarly binds to the immune checkpoint target molecule. In some instances, these non-antibody molecules comprise an extracellular portion of the immune checkpoint target molecule's ligand or binding partner, that is, at least the extracellular portion needed to mediate binding to the immune checkpoint target molecule. In some embodiments this extracellular binding portion of the ligand is joined to additional polypeptide in a fusion protein. In some embodiments the additional polypeptide comprises an Fc or constant region of an antibody.

Programed death-1 (PD-1) is a checkpoint protein on T cells. Antibodies against both PD-1 and its binding partner programmed death-ligand 1 (PD-L1) have been used clinically as immune checkpoint inhibitors (PD-1 blockade). Non-limiting examples of monoclonal antibodies (mAbs) that target PD-1/PD-L1 include: the anti-PD-1 mAbs nivolumab (OPDIVO®, Bristol-Myers Squibb), pembrolizumab (KEYTRUDA®, Merck & Co.), cemiplimab-rwlc (LIBTAYO®, Regeneron Pharmaceuticals), and the anti-PD-L1 mAbs durvalumab (MED14736, IMFINZI™, Medimmune), atezolizumab (MPDL3280A; TECENTRIQ®, Hoffmann-La Roche), avelumab (BAVENCIO®, EMD Serono), and BMS-936559 (Bristol-Myers Squibb). These may be referred to as means for PD-1 blockade, means for inhibiting PD-1/PD-L1 binding, or means for immune checkpoint inhibition.

CTLA-4 is an immune checkpoint molecule expressed on the surface of CD4 and CD8 T cells and on CD25+, FOXP3+ T regulatory (Treg) cells. Non-limiting examples of monoclonal antibodies that target CTLA-4 include ipilimumab (YERVOY®; Bristol-Myers Squibb) and tremelimumab (Medimmune). These may be referred to as means for inhibiting CTLA-4, or means for immune checkpoint inhibition.

TIM-3 (T-cell immunoglobulin and mucin-domain containing-3) is a molecule selectively expressed on IFN- [symbol] -producing CD4⁺ T helper 1 (Th1) and CD8⁺ T cytotoxic 1 (Tc1) T cells. Non-limiting, exemplary antibodies to TIM-3 are disclosed in U.S. Patent Application Publication 20160075783 which is incorporated by reference herein for all it contains regarding anti-TIM-3 antibodies. Other anti-TIM-3 antibodies include TSR-022 (Tesaro). These may be referred to as means for inhibiting TIM-3, or means for immune checkpoint inhibition.

LAG-3 (lymphocyte-activation gene 3; CD223) negatively regulates cellular proliferation, activation, and homeostasis of T cells, in a similar fashion to CTLA-4 and PD-1 and plays a role in Treg suppressive function. Non-limiting exemplary antibodies to LAG-3 include GSK2831781 (GlaxoSmithKline), relatlimab (BMS-986016, Bristol-Myers Squibb), and the antibodies disclosed in U.S. Patent Application Publication 2011/0150892 which is incorporated by reference herein for all it contains regarding anti-LAG-3 antibodies. These may be referred to as means for inhibiting LAG-3, or means for immune checkpoint inhibition.

TIGIT (T cell immunoreceptor with Ig and ITIM domains) is an immunoreceptor inhibitory checkpoint that has been implicated in tumor immunosurveillance. It competes with immune activating receptor CD226 (DNAM-1) for the same set of ligands: CD155 (PVR or poliovirus receptor) and CD112 (Nectin-2 or PVRL2). Anti-TIGIT antibodies have demonstrated synergy with anti-PD-1/PD-L1 antibodies in pre-clinical models. Tiragolumab (Roche), etigilimab (OncoMed), vibostolimab (MK-7684; Merck), and EOS-448 (iTeos Therapeutics) are non-limiting examples of an anti-TIGIT antibodies. They may be referred to as means for inhibiting TIGIT, or means for immune checkpoint inhibition.

GITR (glucocorticoid-induced TNFR-related protein) promotes effector T cell functions and inhibits suppression of immune responses by regulatory T cells. As with OX-40, mentioned above, the checkpoint inhibitor is an agonist of the target, in this case GITR. An agonistic antibody, TRX518 is currently undergoing human clinical trials in cancer. While by itself it may not be sufficient to mediate substantial clinical improvement in advanced cancer, combination with other checkpoint inhibition, such as PD-1 blockade was promising.

Other immune checkpoint inhibitor targets include, but are not limited to, B- and T-cell attenuator (BTLA), CD40, CD122, inducible T-cell costimulator (ICOS), OX40 (tumor necrosis factor receptor superfamily, member 4), Siglec-15, B7H3, CD137 (4-1BB; as with CD40 and OX40, checkpoint inhibition is accomplished with an agonist) and others are potentially useful in the disclosed methods. Several anti-OX40 agonistic monoclonal antibodies are in early phase cancer clinical trials including, but not limited to, MEDI0562 and MEDI6469 (Medimmune), MOXR0916 (Genetech), and PF-04518600 (Pfizer); as is an anti-ICOS agonistic antibody, JTX-2011 (Jounce Therapeutics). Anti-CD40 agonistic antibodies under clinical investigation include dacetuzumab, CP-870,893 (selicrelumab), and Chi Lob 7/4. Anti-siglec-15 antibodies are also known (see, for example, U.S. Pat. No. 8,575,531). Anti-CD137 agonistic antibodies include, but are not limited to, urelumab and utomilumab. Additionally, CD122 has been targeted in cancer clinical trials with bempegaldesleukin (NKTR-214, a pegyltated-IL-2 used as a CD122-biased agonist). B7H3 has been targeted both for immune checkpoint inhibition and as a tumor antigen with reagents such as enoblituzumab, ¹³¹I-omburtamab, ¹⁷⁷Lu-DTPA-omburtamab, ¹³¹I-8H9, ¹²⁴I-8H9, MCG018, and DS-7300a. These may be referred to as means for immune checkpoint inhibition or means for inhibiting (or activating (agonizing), as appropriate) their respective targets.

Compound A therapy may also follow treatment with other types of therapeutic monoclonal antibodies. In some embodiments, the therapeutic monoclonal antibody blocks the so-called “Don't eat me” signal; these include antibodies recognizing ILT2 (for example, BND-22), ILT4 (for example MK-4830), CD47 (for example, Hu5F9-G4), and SIRPα (for example, KWAR23). The Don't-eat-me signal can also be blocked with products such as TTI-621, a fusion protein of the SIRP protein and the antibody Fc region, which act similarly to an anti-CD47 antibody by blocking binding of SIRPα to CD47.

In some embodiments, the therapeutic monoclonal antibody recognizes a cancer antigen, such as, but not limited to, Her2, CD133/prominin, TROP2, claudin 18.2 (for example, claudiximab (IMAB362) or zolbetuximab), CD73/NT5E (for example, MEDI9447, BMS986179, SRF373/NZV930, CPI-006/CPX-006, IPH5301, or TJ004309; CD73/NT5E is also considered an immune checkpoint), crypto-1, or CEACAM5. In some embodiments, the anti-cancer antigen antibody is conjugated to a cytotoxic agent. Non-limiting examples include: SAR408701, an antibody-drug conjugate (ADC) combining a humanized antibody targeting CEACAM5 with the potent cytotoxic maytansinoid derivative DM4; PF-06664178, an antibody-drug conjugate targeting Trop-2 and delivering Aur0101, an auristatin microtubule inhibitor; Sacituzumab govitecan (IMMU-132), an ADC targeting Trop-2 and delivering SN-38; DS-1062a, an ADC targeting Trop-2 and delivering DXd, a topoisomerase 1 inhibitor and derivative of exatecan; and AC133-vcMMAF, a murine anti-human CD133 antibody (AC133) conjugated to a potent cytotoxic drug, monomethyl auristatin.

In some embodiments, the therapeutic monoclonal antibody can be used for M2 macrophage depletion, such as an anti-CD206 antibody. In some embodiments, the anti-CD206 antibody is conjugated to a toxin such as diphtheria toxin.

Pharmaceutically acceptable salts include hydrochloride, sulfate, acetate, phosphate, diphosphate, chloride, maleate, citrate, mesylate, nitrate, tartarate, and gluconate. Various embodiments of Compound A salts are genera comprising these salts, any subset thereof, or any individual species. In some embodiments, the individual Compound A salt is a chloride, a hydrochloride, a sulfate, an acetate, or a phosphate.

### Dosage

The salt form of Compound A dosages are commonly expressed in units of mg/kg, largely for convenience. However, this is actually a rather inaccurate way to estimate drug exposure. When there is a large therapeutic index (for humans, TD₅₀/ED₅₀, the ratio of tolerated dose to effective dose, each for 50% of the population), the variation in drug exposure may be inconsequential. When the therapeutic index is smaller, more precise dosing is needed to attain effectiveness while avoiding unacceptable toxicity. Drug dosages expressed in units of mg/m² can provide this greater precision as drug exposure scales more closely to the subject's body surface area (BSA) than it does to body weight. To achieve this greater precision in Compound A exposure, Compound A dosages are expressed herein in units of mg/m². In the Examples below the Mosteller Formula for BSA was used (BSA (m²)=√height (cm)×weight (kg)/3600), however, other formulae exist, for example, the DuBois & DuBois Formula (BSA (m²)=0.20247×Height(m)^{0.725}×Weight(kg)^{0.425}). These formulae give sufficiently similar results so that any difference is not material.

In various embodiments, the dosage of Compound A is from 0.10 mg/m² to 1.2 mg/m², or 0.15 mg/m² to 1.0 mg/m² (0.2-1.3 mg/m² by mass of the sulfate salt), administered weekly by intravenous infusion. In some embodiments, the dosage does not exceed 0.75, 0.80, 0.90, or 1.0 mg/m². In some embodiments, the dosage is at least 0.15, 0.30, 0.45, or 0.60 mg/m². In some embodiments, the dosage is in a range bounded by any pair of these values. In other embodiments, Compound A is administered weekly, biweekly, every third week, or an interval of any integer number of days between weekly and every third week.

In some embodiments, when used in combination with PD-1 blockade, efficacy can be achieved with a dosage of Compound A of 0.5-0.9 mg/m² (or 0.66-1.2 mg/m² by mass of the sulfate salt).

Estimates of average BSA vary with the population surveyed. By one compilation, the average BSA for men aged 20-79 years in the US was 2.060 m² and 1.830 m² for US women of the same age. Values for UK adult cancer patients were 1.91 m² and 1.71 m² for men and women, respectively, approximately 7% less. Exemplary dose ranges based on the BSA estimates are shown in Table 1.

TABLE 1

| | | | Dose range |
|---|---|---|---|
| Population | Average BSA | | 0.10 mg/m² to 1.2 mg/m² |
| US males, 20-79 | 2.060 | m² | 0.206-2.472 mg |
| US females, 20-79 | 1.830 | m² | 0.183-2.196 mg |
| UK male adult cancer patients | 1.91 | m² | 0.191-2.292 mg |
| UK femail adult cancer | 1.71 | m² | 0.171-2.052 mg |
| patients | | | |

In some embodiments, Compound A, or a pharmaceutically acceptable salt thereof, is administered by intravenous infusion, for example, in 100 mL of normal saline. In order to obtain the proper dose, an appropriate volume of a more concentrated solution would be added to the intravenous solution to provide the required dosage for the individual patient. Thus, the manufactured dosage form can be a solution of Compound A, or a pharmaceutically acceptable salt thereof, at a concentration, for example, of 0.5, 1.0, 1.5, 2.0, 2.5 mg/ml, or in a range bound by any pair of those values. In some embodiments, the solution is contained in a vial (or similar container) filled with 1-2 mL of the solution.

In some embodiments the dosage form comprises Compound A sulfate. In some embodiments the concentration of Compound A sulfate in the dosage form solution is 1.0 to 2.5 mg/mL. In some embodiments the concentration of Compound A sulfate in the dosage form solution is 1.31 mg/ml (equivalent to 1 mg Compound A free base/mL). In some embodiments, the dosage form comprises a 2 mL injection vial containing 1.5 mL of the dosage form solution. In some embodiments, the dosage form solution further comprises 9.1 mg/ml sodium chloride, 0.71 mg/ml sodium citrate dehydrate, sufficient citrate dehydrate or citric acid to adjust the pH to between 4.0 and 5.0 (inclusive), and water for injection to volume.

In establishing a schedule of administration is it usual to attempt to achieve a steady state concentration in the blood stream or other locus in the body, or to at least maintain the concentration of free base form of Compound A above some minimum level. In the present embodiments, Compound A is administered weekly despite the fact that, even at the highest dosages used, Compound A is substantially eliminated from the body within 24 hours. It has been found that this is sufficient to obtain the desired immunotherapeutic effect yet avoids pushing the patient into high grade toxicity due to cytokine release syndrome or other adverse events.

In some embodiments Compound A dosage is adjusted during treatment based on IP-10 induction level. In some embodiments, Compound A dosage is increased if the fold-induction of IP-10 is less than two-fold, five-fold, 10-fold, 20-fold, 30-fold, or 40-fold. In some embodiments, the Compound A dosage adjustment is 0.15 mg/m². In some embodiments, the Compound A dosage adjustment is 0.10 mg/m² or 0.05 mg/m². In some embodiments involving more than one dosage adjustment, the initial dosage adjustment(s) are 0.15 mg/m², but as the desired fold-induction of IP-10 is approached the size of the dosage adjustment is reduced, for example, to 0.10 mg/m² or 0.05 mg/m².

### Toxicity and Adverse Events

The relationship between the efficacy and toxicity of a drug is generally expressed in terms of therapeutic window and therapeutic index. Therapeutic window is the dose range from the lowest dose that exhibits a detectable therapeutic effect up to the maximum tolerated dose (MTD); the highest dose that will the desired therapeutic effect without producing unacceptable toxicity. Most typically therapeutic index is calculated as the ratio of LD₅₀:ED₅₀ when based on animal studies and TD₅₀:ED₅₀ when based on studies in humans (though this calculation could also be derived from animal studies and is sometime called the protective index), where LD₅₀, TD₅₀, and ED₅₀ are the doses that are lethal, toxic, and effective in 50% of the tested population, respectively.

In some embodiments, the dosage of Compound A avoids or reduces the severity or incidence of toxicity and related adverse events. In various aspects of these embodiments the toxicity is an observable toxicity, a substantial toxicity, a severe toxicity, or an acceptable toxicity, or a dose-limiting toxicity (such as but not limited to a MTD). By an observable toxicity it is meant that while a change is observed the effect is negligible or mild. By substantial toxicity it is meant that there is a negative impact on the patient's overall health or quality of life. In some instances, a substantial toxicity may be mitigated or resolved with other ongoing medical intervention. By a severe toxicity it is meant that the effect requires acute medical intervention and/or dose reduction or suspension of treatment. The acceptability of the toxicity will be influenced by the particular disease being treated and its severity and the availability of mitigating medical intervention.

Toxicities and adverse events are sometimes graded according to a 5-point scale. A grade 1 or mild toxicity is asymptomatic or induces only mild symptoms; may be characterized by clinical or diagnostic observations only; and intervention is not indicated. A grade 2 or moderate toxicity may impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) may be impaired. Grade 4 toxicities are life-threatening and urgent intervention is indicated. Grade 5 toxicity produces an adverse event-related death. Thus, in various embodiments, use of Compound A in the herein disclosed regimen, or at a specified dosage reduces the grade of a toxicity associated with treatment by at least one grade as compared to use of the drug according to another regimen. In other embodiments, by use of Compound A in a specified regimen or at a specified dosage, a toxicity is confined to grade 2 or less, grade 1 or less, or produces no observation of the toxicity.

In some embodiments, Compound A dosage is reduced if an unacceptable level of toxicity is observed. In some embodiments, the Compound A dosage is reduced 0.15 mg/m². In some embodiments, the Compound A dosage is reduced 0.10 mg/m² or 0.05 mg/m². In some embodiments, infusion time is increased to mitigate adverse reactions.

### Cytokine Induction

The herein described Compound A monotherapy induces expression of several cytokines, most notably the pro-inflammatory cytokines IFN-γ and IP-10. As used herein, induction refers to an increase in expression that is ≥2-fold the baseline level of expression before initiation of Compound A monotherapy. In alternative embodiments, induction refers to an increase in expression that is ≥5-fold the baseline level of expression before initiation of Compound A monotherapy. In some embodiments, fold induction is based on IP-10 AUC. In some embodiments, IP-10 induction is based on Cmax.

Observations indicate that greater levels of IP-10 induction was associated with prior immune checkpoint inhibition, specifically PD-1 blockade therapy (with either anti-PD-1 or anti-PD-L1). Greater IP-10 induction was also associated with prior chemotherapy, specifically DNA-damaging chemotherapy. Greater IP-10 induction was also associated with prior targeted therapies. Additionally, higher baseline levels of IP-10 may indicate a higher probability of response via further IP-10 induction to Compound A monotherapy.

In some embodiments, cytokine induction is monitored during Compound A monotherapy. Monitoring can include assaying plasma or serum levels of cytokines, such as IFN-γ and IP-10, or IFN-γ inducible gene products, such as CXCL10 (IP-10) or CXCL11. Monitoring can include quantification of whole blood RNA transcripts encoding these proteins. A baseline reading is established before the first infusion of Compound A monotherapy and samples can be collected as specified time points thereafter. (Preferably, the sample for the baseline reading is obtained within 1 to with 6 hours before starting the infusion, but anytime within 24 hours before is acceptable.) Cytokine induction can be assessed after the first infusion of Compound A, after the second infusion of Compound A, after 6 weeks of treatment, at the time of the first efficacy evaluation scan, at the time of disease progression, at the time of clinical response, at the time of an adverse event, or on a regular schedule, for example, every 3 or 6 weeks, counting from either initiation of treatment or from a first measurement after initiation of treatment. Depending on the change in cytokine level, for example IP-10 level, Compound A dosage could then be changed for the next infusion to either increase the probability of response and/or decrease the risk of severe adverse events.

### Importance of IP-10 Induction to Immune Response and How IP-10 Induction Correlates with Response in Cancers

IP-10 is known for exerting antitumor immunity by binding CXCR3, which is expressed on immune cells such as monocytes, CD4⁺ Th1 T cells, effector CD8⁺ T cells, NK, and dendritic cells. IP-10 is inducible by both IFN-γ and type I interferons and is produced by CD4⁺ T cells, NK/NKT cells, monocytes, dendritic cells, fibroblasts, endothelial, and epithelial cells. IP-10 induction triggers CXCR3-mediated Th1 polarization, which in turn facilitates the maturation and activation of cytotoxic T lymphocytes, NK, and macrophages and their migration to the tumor microenvironment. IP-10 induction can also activate CXCR3⁺ CD4⁺/CD8⁺ T cells to enhance antitumor immunity.

In contrast, IP-10 bound to CXCR3 expressed on cancer cells can promote survival and metastasis through autocrine signaling. Enhanced IP-10-CXCR3 signaling in the tumor microenvironment is considered a negative predictor of response whereas IP-10-CXCR3 paracrine signaling axis in immune cells elicit antitumor immune response. Therefore, IP-10 induction in the blood may be predictive for paracrine signaling for antitumor immunity; however, migration of activated immune cells and CXCR3 expression in the tumor microenvironment must be assessed to fully correlate IP-10 induction with antitumor response.

An IFN-γ-mediated gene signature, which includes IP-10, is a well-studied predictive biomarker for response in cancer patients. IFN-γ/IP-10 induction was associated with increased antitumor immunity, leading to clinical response in various solid tumors. Interestingly, these studies involved immunomodulatory agents such as anti-PD-(L)1 mAb, anti-CTLA-4 mAb, IFN-α, poly-I:C, and a DNA vaccine to elicit antitumor immunity via IFN-γ/IP-10 induction. Additionally, PD-1 and CTLA-4 blockade induced macrophage-derived IP-10, which correlated with antitumor immune response. In other instances where IP-10 was induced in stromal cells or following chemoradiation, recruitment of Tregs was favored to suppress immune activities. Thus, the role of IFN-γ/IP-10 induction as immune activating versus suppressing may depend on the tumor and treatment types (e.g., immunotherapy versus chemotherapy) that may shift the balance between Treg and Th1-mediated immune cells.

### Method of Treatment

Provided herein are methods of treating cancer in a mammal, for example, a human, comprising administering Compound A by intravenous infusion as monotherapy. Dosage is specified in units of mg/m², in order to more precisely control patient exposure to the Compound A. As described above, dosage can be in the range of 0.10 mg/m² to 1.2 mg/m². This amount of drug is typically infused over 20-90 minutes, for example 30 minutes, or 60 minutes or any range bound by a pair of these values. Compound A will be administered by infusion 1-6 times over a period of 3 to 6 weeks, with administration not occurring more often than once per week. It can be convenient to refer to such a unit of treatment as a “cycle”, however, there is no necessary event tied to the ending of one cycle or the beginning of the next. Indeed, treatment can and often will continue uninterrupted according to the schedule of the cycle until 1) the disease (cancer) progresses and the drug is stopped due to it not achieving its desired effect, 2) the patient has toxicity from the and needs to discontinue treatment, 3) the patient and/or treating physician determines that another treatment would be better for the patient, or 4) all disease (cancer) has disappeared and the patient and/or treating physician decide to stop therapy. In some instances it can be appropriate to adjust the dosage in response to ineffectiveness or toxicity instead of discontinuing treatment, as described above.

Compound A monotherapy after a prior therapy can commence any time after the last administration of the prior therapy and the decision to adopt a new therapeutic approach is made. In some embodiments, Compound A therapy commences after a rest period to allow the amount of the prior therapy drug in the body to become substantially reduced or eliminated, to recover from surgery, or for any adverse events produced by the prior therapy to resolve. In various embodiments, the interval between the last administration of the prior therapy and commencing Compound A monotherapy is at least 2, 3, or 4 weeks, or a month. In some embodiments, Compound A monotherapy does not commence until toxicity from the prior therapy has reduced to grade 1 or less, or to baseline. In some embodiments, the prior therapy caused permanent damage to an organ and Compound A monotherapy is not initiated until mitigating therapy for the impaired or lost function of the damaged organ is established. For example, the thyroid gland or pituitary gland may be damaged and hormone replacement therapy undertaken. In one aspect of these embodiments, the prior therapy was immunotherapy, for example, immune checkpoint inhibition therapy. In another aspect of these embodiments the prior therapy was chemo therapy, for example cytotoxic or DNA-damaging chemotherapy.

Observations indicate that prior treatment can bias the response to Compound A monotherapy. For example, prior PD-1 blockade immune checkpoint inhibition was associated with greater IP-10 induction and better clinical response. Thus, in some embodiments, Compound A monotherapy is used to treat patients coming off of immune checkpoint inhibition therapy (for example, due to progressive disease or unacceptable toxicity). In one aspect of these embodiments, the patients have also had prior treatment with chemotherapy, for example, cytotoxic or DNA-damaging chemotherapy. In some of these embodiments, Compound A monotherapy is commenced within 12 weeks or within 24 weeks (or within 3 months or within 6 months) of the last dose of the prior therapy, or within 12 weeks or within 24 weeks (or within 3 months or within 6 months) of the last dose of the prior immune checkpoint inhibition therapy. In some embodiments, the prior immune checkpoint inhibition therapy, was anti-PD-1 therapy, anti-PD-L1 therapy, or either. In some embodiments, the prior immune checkpoint inhibition therapy, was anti-CTLA-4 therapy. In still further embodiments, the prior immune checkpoint inhibition therapy was anti-LAG-3, anti-Siglec-15, anti-TIGIT, oranti-TIM-3. In further embodiments, the prior immune checkpoint inhibition therapy can be anti-B7H3, anti-CD137, anti-OX40, anti-CD40, anti-CD122, anti-ICOS, or anti-CD73/NT5E.

In other embodiments, immune checkpoint inhibition therapy and treatment with Compound A are undertaken as sequential treatments, in which at least 1-3 doses of the immune checkpoint inhibitor are administered before treatment with Compound A commences. In some embodiments, immune checkpoint inhibition therapy is discontinued before commencing treatment with Compound A. In other embodiments, administration of the immune checkpoint inhibitor continues concurrently with treatment with Compound A. In aspects of these embodiments, the immune checkpoint inhibitor is administered according to its usual schedule.

In various embodiments, a window during which Compound A monotherapy or sequential therapy is initiated is defined by any of the above teachings regarding earliest administration and periods within which it should be commenced.

The effectiveness of treatment can be monitored over the course of treatment. Monitoring can be accomplished by imaging the tumor(s), for example, by simple X-ray, CAT or CT scan, PET scan, or MRI. In some embodiments, these scanning technologies can be referred to as means for imaging, and taking such a scan can be referred to as a step for imaging.

The effectiveness of cancer therapy is typically measured in terms of “response.” The techniques to monitor responses can be similar to the tests used to diagnose cancer such as, but not limited to:
A lump or tumor involving some lymph nodes can be felt and measured externally by physical examination.
Some internal cancer tumors will show up on an x-ray, CT scan, PET scan, CT/PET scan or MRI and can be measured with a ruler.
Blood tests, including those that measure organ function can be performed.
A tumor marker test can be done for certain cancers.

Regardless of the test used, whether blood test, cell count, or tumor marker test, it is repeated at specific intervals so that the results can be compared to earlier tests of the same type.

Response to cancer treatment is defined several ways:
Complete response—all of the cancer or tumor disappears; there is no evidence of disease. Expression level of tumor marker (if applicable) may fall within the normal range.
Partial response—the cancer has shrunk by a percentage, but disease remains. Levels of a tumor marker (if applicable) may have fallen (or increased, based on the tumor marker, as an indication of decreased tumor burden) but evidence of disease remains.
Stable disease—the cancer has neither grown, nor shrunk; the amount of disease has not changed. A tumor marker (if applicable) has not changed significantly.
Disease progression—the cancer has grown; there is more disease now than before treatment. A tumor marker test (if applicable) shows that a tumor marker has risen.

Other measures of the efficacy of cancer treatment include intervals of overall survival (that is time to death from any cause, measured from diagnosis or from initiation of the treatment being evaluated)), cancer-free survival (that is, the length of time after a complete response cancer remains undetectable), and progression-free survival (that is, the length of time after disease stabilization, partial response, or complete response that resumed tumor growth is not detectable).

There are two standard methods for the evaluation of solid cancer treatment response with regard to tumor size (tumor burden), the WHO and RECIST standards. These methods measure a solid tumor to compare a current tumor with past measurements or to compare changes with future measurements and to make changes in a treatment regimen. In the WHO method, the solid tumor's long and short axes are measured with the product of these two measurements is then calculated; if there are multiple solid tumors, the sum of all the products is calculated. In the RECIST method, only the long axis is measured. If there are multiple solid tumors, the sum of all the long axes measurements is calculated. However, with lymph nodes, the short axis is measured instead of the long axis.

Beneficial associations between clinical benefit and a particular pharmacokinetic profile for Compound A, based on the concentration of the free base in plasma, have been observed. A Cmax in plasma of Compound A free base of 8.3 ng/mL and/or an area under the curve (AUC) in plasma of Compound A free base of 3.4 ng/mL*Day is associated with greater likelihood of clinical benefit from monotherapy with Compound A. Thus some embodiments are methods of treating cancer comprising administering an therapeutically effective amount of Compound A, or a pharmaceutically acceptable salt thereof, to the patient to provide a plasma concentration profile in said patient comprsing: a maximum plasma concentration (Cmax) of Compound A free base of >8.3 ng/mL, an AUC of Compound A free base of >3.4 ng/mL*Day, or both. In some embodiments, the In further embodiments, the likelihood of clinical benefit is increased as compared to treatment with Compound A that does not attain the plasma concentration profile. In other embodiments, the Cmax profile attained is >5, >6, >7, >8, >9, >10, >11, or >12 ng/mL. In other embodiments, the AUC profile attained is >1, >2, >3, >4, or >5 ng/mL*Day.

A Cₘₐₓ in plasma of Compound A free base of 7.6 ng/mL and/or an AUC in plasma of Compound A free base of 2.2 ng/mL*Day is associated with greater likelihood of clinical benefit from combination therapy with Compound A and an anti-PD1 antibody. Thus some embodiments are methods of treating cancer comprising administering an therapeutically effective amount of Compound A, or a pharmaceutically acceptable salt thereof, and an anti-PD-1 antibody to the patient to provide a plasma concentration profile in said patient comprsing: a maximum plasma concentration (Cmax) of Compound A free base of 7.6 ng/mL, an area under the curve (AUC) of Compound A free base of 2.2 ng/mL*Day, or both. In further embodiments, the likelihood of clinical benefit is increased as compared to treatment with Compound A that does not attain the plasma concentration profile. In other embodiments, the Cmax profile attained is >5, >6, >7, >8, >9, or >10 ng/mL. In other embodiments, the AUC profile attained is >1, >2, >3, >4, or >5 ng/mL*Day.

A Cₘₐₓ in plasma of Compound A free base of 10.5 ng/mL and/or an AUC in plasma of Compound A free base of 2.1 ng/mL*Day is associated with greater likelihood of clinical benefit from combination therapy with Compound A and an anti-PD-L1 antibody. Thus some embodiments are methods of treating cancer comprising administering an therapeutically effective amount of Compound A, or a pharmaceutically acceptable salt thereof, and an anti-PD-L1 antibody to the patient to provide a plasma concentration profile in said patient comprsing: a maximum plasma concentration (Cₘₐₓ) of Compound A free base of 10.5 ng/mL, an area under the curve (AUC) of Compound A free base of 2.1 ng/mL*Day, or both. In further embodiments, the likelihood of clinical benefit is increased as compared to treatment with Compound A that does not attain the plasma concentration profile. In other embodiments, the Cmax profile attained is >5, >6, >7, >8, >9, >10, >11, or >12 ng/mL. In other embodiments, the AUC profile attained is >1, >2, >3, >4, or >5 ng/mL*Day.

In particular embodiments, “clinical benefit” means complete response, partial response, or stable disease >18 weeks according to RECIST version 1.1.

The term “treating” or “treatment” broadly includes any kind of treatment activity, including the diagnosis, mitigation, or prevention of disease, or aspect thereof, in man or other animals, or any activity that otherwise affects the structure or any function of the body of man or other animals. Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. In some embodiments, the orders, instructions, and advice aspect of treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians group, and the like. All such orders, instructions, and advice are to be seen as conditioning receipt of the benefit of the treatment on compliance with the instruction. In some instances, a financial benefit is also received by the patient for compliance with such orders, instructions, and advice. In some instances, a financial benefit is also received by the healthcare professional for compliance with such orders, instructions, and advice.

Non-limiting examples of cancers which can be treated by the disclosed methods may include, but are not limited to, acute lymphoblastic leukemia, acute myeloid leukemia, adrenocortical carcinoma, AIDS-related lymphoma, an AIDS-related malignancy, anal cancer, bile duct cancer, bladder cancer, bone cancer, brain stem glioma, a brain tumor (e.g., astrocytoma, cerebellar astrocytoma; cerebral astrocytoma/malignant glioma, ependymoma brain tumor, supratentorial primitive brain tumor, a neuroectodermal tumor, visual pathway and hypothalamic glioma, etc.), breast cancer, a bronchial adenoma/carcinoid, carcinoid tumor, carcinoma (adrenocortical, gastrointestinal, islet cell, skin, unknown primary, etc.); cervical cancer, a childhood cancer, chronic lymphocytic leukemia, chronic myelogenous leukemia, a chronic myeloproliferative disorder, clear cell sarcoma of tendon sheaths, colon cancer, colorectal cancer, cutaneous T-cell lymphoma, endometrial cancer, ependymoma, epithelial cancer, esophageal cancer, a Ewing family of tumor, extracranial germ cell tumor, extragonadal germ cell tumor, extrahepatic bile duct cancer, eye cancer, intraocular melanoma, gallbladder cancer, gastric cancer, gastrointestinal carcinoid tumor, ovarian germ cell tumor, gestational trophoblastic tumor, hairy cell leukemia, head and neck cancer, hepatocellular cancer, Hodgkin's lymphoma, hypopharyngeal cancer, islet cell carcinoma (endocrine pancreas), Kaposi's sarcoma, kidney cancer, laryngeal cancer, lip and oral cavity cancer, primary liver cancer, lung cancer, non-small cell lung cancer, small cell lung cancer, primary central nervous system lymphoma, Non-Hodgkin's lymphoma, Waldenstrom's macroglobulinemia, malignant mesothelioma, malignant thymoma, medulloblastoma, melanoma, Merkel cell carcinoma, primary metastatic squamous neck cancer, multiple endocrine neoplasia syndrome, multiple myeloma/plasma cell neoplasm, mycosis fungoides, a myelodysplastic syndrome, multiple myeloma, nasal cavity and paranasal sinus cancer, nasopharyngeal cancer, neuroblastoma, oral cancer, oropharyngeal cancer, ovarian epithelial cancer, ovarian low malignant potential tumor, pancreatic cancer, parathyroid cancer, pheochromocytoma penile cancer, pineal and supratentorial primitive neuroectodermal tumors, pituitary tumor, pleuropulmonary blastoma, prostate cancer, rectal cancer, renal cancer, transitional cell cancer, retinoblastoma, rhabdomyosarcoma, salivary gland cancer, sarcoma (e.g., Ewing's family of tumors, Kaposi's sarcoma, osteosarcoma/malignant fibrous histiocytoma of bone, soft tissue, etc.), Sezary syndrome, skin cancer, small intestine cancer, testicular cancer, thymoma, thyroid cancer, trophoblastic tumor, vaginal cancer, vulvar cancer, or Wilms' Tumor. Some embodiments are methods or treating a solid tumor. Some embodiments are methods of treating a carcinoma, or a sarcoma, or a hematologic malignancy.

Each of the herein disclosed methods of treatment may be expressed as a corresponding composition for use in treating cancer, use of a composition in treating cancer, or use of a composition in the manufacture of a medicament for treating cancer, etc.

### Formulation

Pharmaceutical compositions comprising Compound A can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor.

Liquid formulations suitable for infusion may comprise physiologically acceptable sterile aqueous or non-aqueous solutions, dispersions, suspensions or emulsions and sterile powders for reconstitution into sterile infusable solutions or dispersions. Examples of suitable aqueous and non-aqueous carriers, diluents, solvents or vehicles include, but are not limited to, water, ethanol, polyols (propylene glycol, polyethyleneglycol (PEG), glycerol, and the like), suitable mixtures thereof, vegetable oils (such as olive oil), polymers, liposomes, nanoparticles, nanomicellar formulation, pegylation, aluminum gel, associated proteins or polypeptides such as albumins, and injectable organic esters such as ethyl oleate. Proper fluidity can be maintained, for example, by the use of a coating such as lecithin, by the maintenance of the required particle size in the case of dispersions and by the use of surfactants.

A pharmaceutical composition of Compound A can optionally include a pharmaceutically acceptable carrier that facilitates processing of the active compound into pharmaceutically acceptable compositions. Such a carrier generally is mixed with an active compound or permitted to dilute or enclose the active compound. Any of a variety of pharmaceutically acceptable carriers can be used including, without limitation, aqueous media such as, e.g., water, saline, glycine, hyaluronic acid and the like; solvents; dispersion media; polymers, liposomes, nanoparticles, nanomicellar formulation, pegylation, aluminum gel; associated proteins or polypeptides such as albumins; antibacterial and antifungal agents; isotonic and absorption delaying agents; or any other inactive ingredient.

In some embodiments, the dosage appropriate amount of Compound A based on the patient's BSA is diluted into 100 ml normal saline for infusion.

In some embodiments, Compound A injectable solution is a sterile, preservative-free, colorless solution containing Compound A and inactive ingredients, sodium chloride and sodium citrate, for intravenous use. The primary packaging consists of a 2-mL glass vial containing 1.5 mL of Compound A Injectable Solution. The concentration of Compound A free base can be 1.0 mg/ml (although the solution may be made by dissolution of a pharmaceutically acceptable salt, such as the sulfate salt) and the volume of the drug solution in the vial to be added to 100 mL normal saline is calculated based on 1.0 mg/mL in the vial and the mass or BSA of the pateint. Directions can be provided in a pharmacy manual or prescribing information (package insert) for dosing solution preparation and infusion requirements.

In some embodiments, Compound A injectable solution vials are for single use only and can be supplied in a 2 ml clear vial. The vial is capped with a 13-mm rubber stopper and sealed with a 13-mm plastic aluminum seal. An aluminum seal with a plastic flip-off cap can be used to secure the closure in place. Vials can be shipped in small cardboard cartons containing a plurality of vials, for example six vials each. The vials are to be stored at room temperature (15°-25° C.) and protected from light in a secure, temperature controlled, limited access area. The final compound A sulfate salt concentration can be about 1.3 mg/mL This is equivalent to a free base of approximately 1.0 mg/mL.

### EXAMPLES

The following non-limiting examples are provided for illustrative purposes only in order to facilitate a more complete understanding of representative embodiments now contemplated. These examples should not be construed to limit any of the embodiments described in the present specification,

### Example 1

### Compound A Pharmacokinetics and Safety

A multi-center, open-label, dose escalation/dose expansion Phase 1 study of a Compound A sulfate salt, as a single agent, in subjects with histologically confirmed, unresectable or metastatic solid tumors that have relapsed or are refractory to standard therapies or for whom there is no approved therapy is under way. Subjects are administered the Compound A salt in 100 ml normal saline as an intravenous infusion. A treatment cycle is 21 days with 3 weekly infusions on days 1, 8, and 15.

The ongoing Phase 1 dose-escalation trial has so far enrolled 36 subjects treated with at least one dose of the Compound A salt as monotherapy. Subject population summary (Table 2) shows the demographics, and prior lines of therapy recorded at screening. (A line of therapy consists of complete cycle of a single agent, a regimen consisting of a combination of several drugs, or a planned sequential therapy of various regimens.) Enrolled subjects had 15 solid tumor types with most frequent being NSCLC, endometrial carcinoma/sarcoma, and ovarian cancer.

TABLE 2
Summary of treated subject population at baseline

Population (N = 36)

Demographics

| | | |
|---|---|---|
| Age (years), median [range] | 66 | [33-88] |
| Female, n (%) | 24 | (66.7) |
| Male, n (%) | 12 | (33.3) |

Prior lines of therapy

| | | |
|---|---|---|
| Median [Range] | 4 | [0-13] |

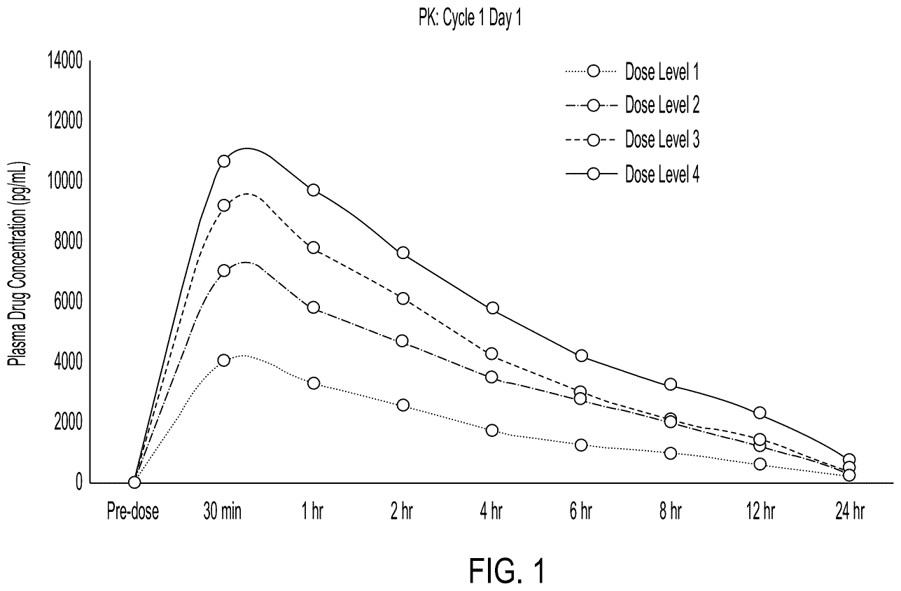

Compound A dosages were initially specified in mg/kg, but proved problematic.Dosages based on body surface area (BSA)-based dosages (mg/m²) were adopted to account for intersubject variability in weight and drug exposure. The Mosteller Formula was used: BSA=√height (cm)×weight (kg)/3600. Five escalating dosages (0.30 mg/m², 0.45 mg/m², 0.60 mg/m², 0.75 mg/m², and 0.90 mg/m²) were administered intravenously, and a dose-dependent increase in Compound A free base exposure was observed with an approximate half-life of 4 hours (FIG. 1). Compound A was effectively cleared from the bloodstream by about 24-hours post-infusion for all dosages and no signs of drug accumulation from Cycle 1 Day 1 to Day 8 were observed.

There was no apparent dose-dependent increase in toxicity in this trial. Compound A monotherapy was well-tolerated overall with no Grade 4 or 5 adverse events (AE's) and 11 of 36 (31%) subjects did not experience any drug-related AE's. Most common drug-related AE's were associated with immune activation.

Fold of IP-10 induction in patients was not observed to be correlated with safety profile, as the overall safety profile was comparable among subjects with substantial IP-10 induction versus subjects with minimal IP-10 induction (Table 3). However, it has been demonstrated that IP-10 induction correlates with clinical benefit to patients receiving Compound A monotherapy.

TABLE 3
Safety profile of subjects with or without IP-10 induction

| | IP-10 induction, >5- | No IP-10 induction, <5- |
|---|---|---|
| | fold increase (n = 17) | fold increase (n = 19) |
| No AE | 4 (24%) | 7 (37%) |
| Grade 1 AE | 4 (24%) | 6 (32%) |
| Grade 2 AE | 7 (41%) | 5 (26%) |
| Grade 3 AE | 2 (12%) | 1 (5%) |

IP-10 induction (>5-fold increase) was more likely in subjects with prior anti-PD-(L)1 mAb and/or chemotherapy treatments (Table 3); however, such immune activation favored clinical response in subjects with prior anti-PD-(L)1 mAb treatment.

### Example 2

### Cytokine Biomarker Assays

For each subject treated with at least one dose of Compound A salt monotherapy, blood samples were collected on Cycle 1 Day 1, Cycle 1 Day 8, and Cycle 3 Day 1 at pre-dose, and post-infusion at 0.5, 1, 2, 4, 6, 8,12, and 24 hours (only at pre-dose and 1- and 4- hours post-infusion on Cycle 3 Day 1). Pharmacodynamic analysis of Compound A included cytokine quantification and whole-blood RNA transcriptional profiling.

Collected plasma samples were quantified for cytokine biomarkers implicated in inflammatory responses, immune system regulation, and other biological processes. Assays for cytokine quantification were performed by Frontage Laboratories, Inc. utilizing Meso Scale Discovery (MSD) V-PLEX and U-PLEX plates pre-coated with capture antibodies for IL-5, IL-17A, IFN-γ, IL-2, IL-6, IL-8, IL-10, IL-12p70, TNF-α, IFN-α, IFN-β, and IP-10. Plasma samples were added directly to the plate to allow the analytes in the samples to bind to the capture antibodies. Electrochemiluminiscent label (MSD SOLFO-TAG™)-conjugated detection antibodies were then added to visualize the plates by a MSD plate reader. The luminescence intensity was measured to quantify the amount of captured analytes (or cytokine levels) in each sample.

RNA extracted from whole-blood samples was analyzed via the NanoString nCounter platform of high-plex assay designed to quantify gene expression using the pre-designed Human Pan Cancer Immune Profiling panel of genes. Intra-patient comparisons among various time points was performed. The relative transcript levels of selected genes were analyzed based on the data from cytokine quantification.

Handling of and laboratory analysis of subject samples were performed in compliance of Good Clinical Practice (GCP) and Good Laboratory Practice (GLP).

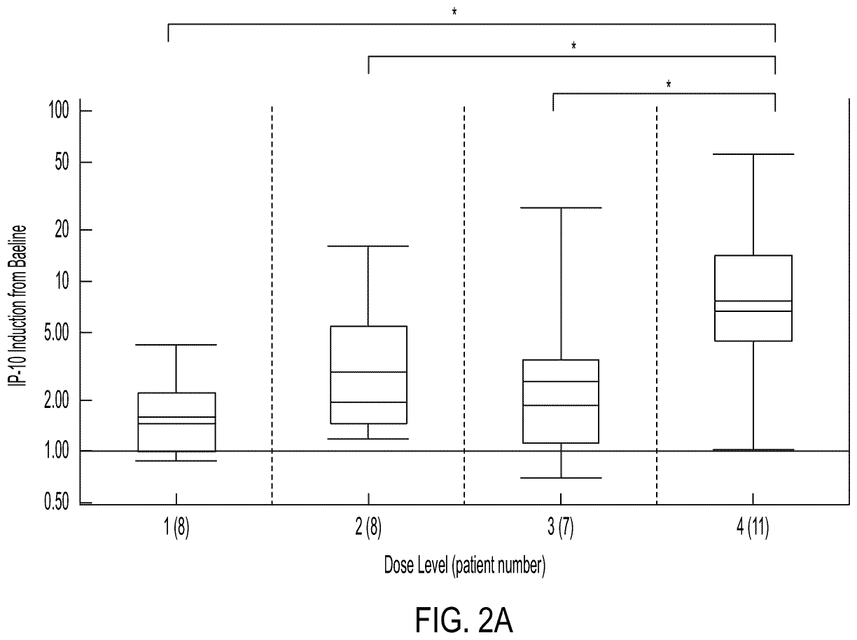

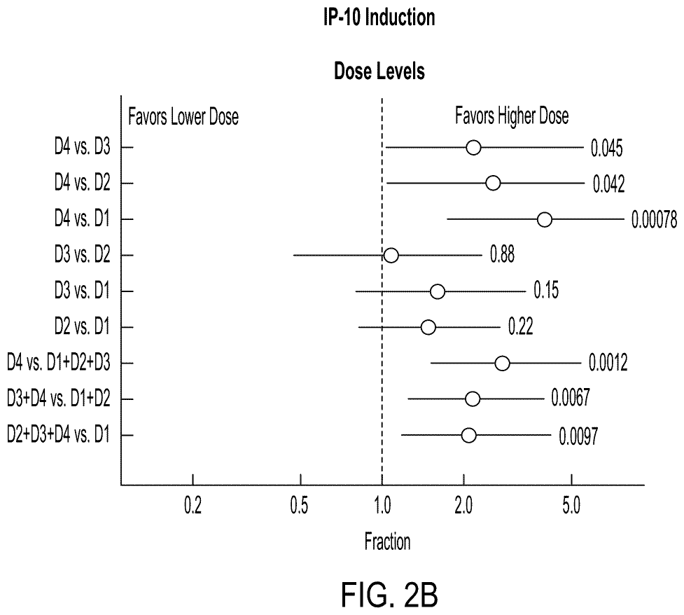

Data analysis of cytokine quantification revealed that Compound A monotherapy significantly increased blood IP-10 levels (FIG. 2A) and IFN-γ (data not shown). The most robust cytokine induction was observed in response to the highest Compound A dose level tested, 0.75 mg/m² dose level 4. The increase in IFN-γ and IP-10 levels strongly suggests that Compound A upregulates the interferon-inducible gene signature, a well-known marker for antitumor immunity. Compound A-induced IP-10 and IFN-γ expression, and increase in interferon-inducible gene transcripts, correlated with clinical response (see Example 4).

### Example 3

### Cytokine Pharmacodynamics in Response to Doses of Compound A Salt

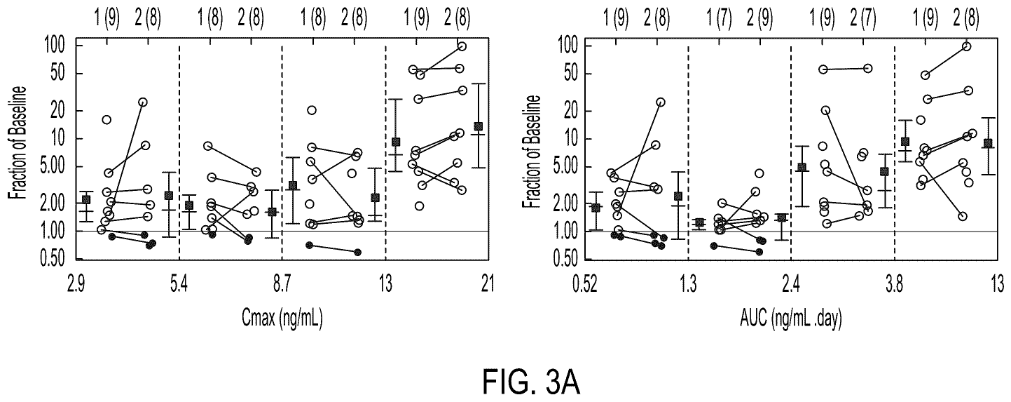

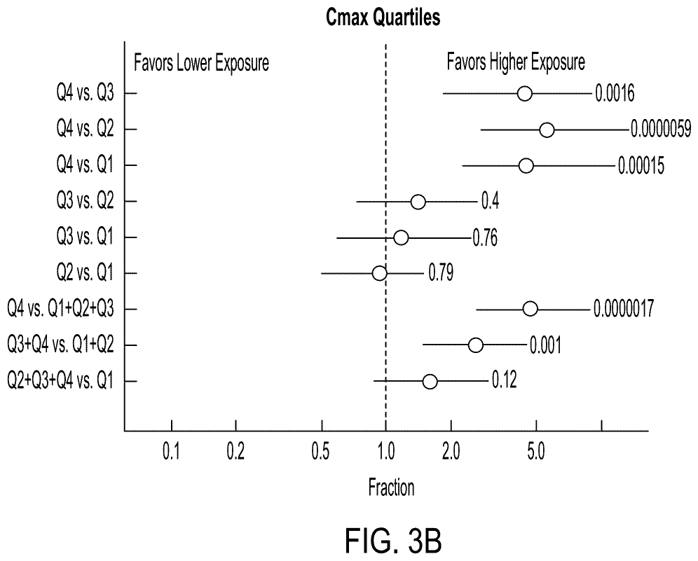

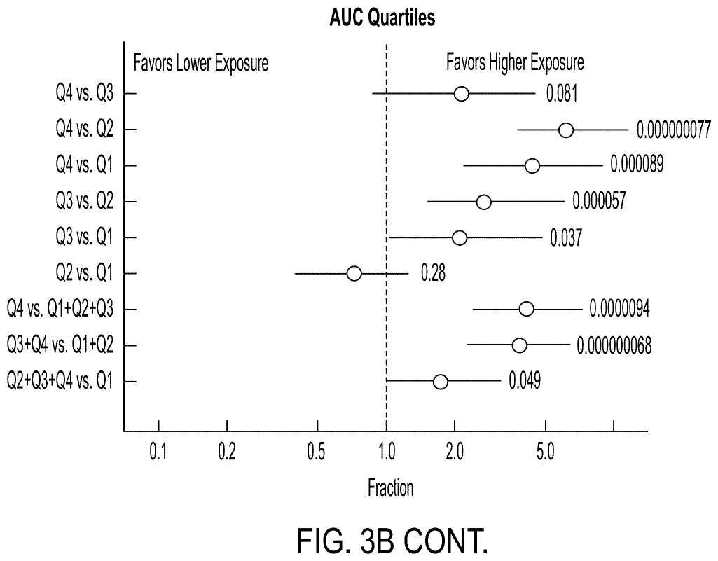

To assess the influence of intravenous administration of Compound A salt on cytokine production, plasma levels of IFN-α, IFN-β, IFN-γ, IL-2, IL-5, IL-6, IL-8, IL-10, IL-12, IL-17, interferon inducible protein 10 (IP-10) and TNF-α were quantified using a commercially available MSD V-plex assay (Meso Scale Discoveries) per manufacturer's instructions. Plasma samples were collected at Cycle 1, Cycle 3, and Cycle 6. Of the cytokines quantified, IP-10, IFN-γ, IL-6, IL-8, IL-10, and TNF-α had detectable levels. Peak cytokine levels were normalized to Cycle 1 Day 1 baseline for each subject and compared based on Compound A dose levels (Table 4). The greater peak cytokine level from either Cycle 1 Days 1 or 8 were selected for the analysis to be inclusive of all subjects. There was a dose-dependent increase in IP-10 and IFN-γ, indicating immune activation by Compound A. Medians and ranges of peak cytokine levels/baseline from each dose level are shown in Table 4. Cytokine induction also correlated to peak plasma concentration of Compound A (FIG. 3).

TABLE 4
Relationship between dose level and cytokine levels
(median peak level relative to baseline level)

| | Dose Level 1 | Dose Level 2 | Dose Level 3 | Dose Level 4 |
|---|---|---|---|---|
| Cytokine | 0.30 mg/m² | 0.45 mg/m² | 0.60 mg/m² | 0.75 mg/m² |
| IP-10 | 1.99 | 5.67 | 6.58 | 13.47 |
| IFN-γ | 2.04 | 2.96 | 3.97 | 5.23 |

It was clear that IP-10 and IFN-γ inductions were most robust at the higher Compound A salt dose (Table 4), and such IP-10 and IFN-γ inductions were statistically significant (p=0.002 and p=0.013, respectively). Dose-dependent increase in IP-10 transcripts was also observed in whole-blood RNA (data not shown). Additionally, a dose-dependent increase in IP-10 induction was observed even when IP-10 changes from baseline were shown in AUEC levels (FIG. 2A). Compound A dose-dependent increase in IP-10 with the highest IP-10 induction at 0.75 mg/m² (D4; Dose Level 4) was statistically significant as seen in FIG. 2B.

Median IP-10/IFN-γ changes are higher in partial response and prolonged stable disease (SD) subjects (data not shown), indicating that IP-10 and IFN-γ induction correlates with clinical response to Compound A. This was confirmed by RNA transcriptional profiling via Nanostring analysis, which showed increases in IFN-inducible gene transcript levels for CXCL10, CXCL11, and Indoleamine 2,3-dioxygenase (IDO1) in responders (partial response and stable disease >18 weeks) (data not shown). The window of immune activation necessary for response could be defined by IP-10 induction of 5- to 40-fold increase, which was seen in PR (2 of 2) and prolonged SD subjects (6 of 7). An out of window induction of IP-10 higher than 100-fold increase may instead require dose reduction to minimize the risks of toxicity such as cytokine release syndrome.

### Example 4

### Efficacy of Compound A Salt Monotherapy

A disease control rate of 63% (2 partial response [PR]+18 stable disease [SD]) was achieved according to irRECIST. 7 subjects (22%) had prolonged disease control (study duration >18 weeks), one of whom who had the longest study duration of 68 weeks on Compound A salt monotherapy treatment. There were 12 subjects with progressive disease (PD, 38%) as best response. Both partial response subjects received the higher dose of Compound A salt at 0.75 mg/m².

### Example 5

### Correlation of Compound A salt Pharmacokinetics, Pharmacodynamics, Marker of IP-10 Induction, and Clinical Efficacy

An analysis was performed to investigate pharmacokinetic parameters of the free base form of Compound A and their relation to plasma IP-10 induction levels from baseline and correlation as with clinical response. Response was defined by best overall response—immune-related partial response (irPR), immune-related stable disease (irSD) with study duration over or under 18 weeks, and unconfirmed immune-related progressive disease (uirPD). Clinical benefit was defined as either partial response (irPR) or irSD >18 weeks. IP-10 cytokine induction levels of 32 patients evaluable for efficacy, were analyzed with respect to dosage in Cycle 1. Because of inter-patient variabilities of ADME (absorption, distribution, metabolism, and excretion) of individual patients, patients receiving the same dosage may have very different Compound A (measured as free base) AUC or Cmax values, which in turn will produce very different downstream pharmacodynamic effects, such as IP-10 induction. For example, patient A dosed with dose level 2 with slower metabolism may have higher AUC and/or Cmax than patient B who was dosed with dose level 3, as a result patient A may produce higher level of IP-10 induction from baseline. Ultimately, it can be appropriate to adjust Compound A dosage based on pharmacokinetic (AUC or Cmax) or pharmacodynamics (e.g., IP-10 induction) factors.

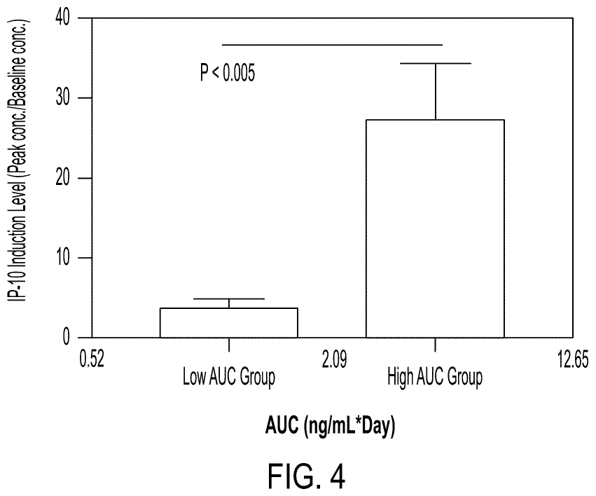

As shown in FIG. 4, IP-10 induction level, presented as the induction from baseline, divided into two groups in accordance with the AUC for Compound A , with 16 patients in a low AUC group (AUC below 2.09 ng/mL*Day) and 16 patients in the ahigh AUC group (AUC above 2.09 ng/mL*Day) groups respectively. The mean value of induction level of IP-10 is 3.68-fold in the low AUC group but 27.2-fold in the high AUC group, indicating that a minimum of AUC 2.09 ng/ng/mL*Day is required to induce IP-10 that in turn results in clinical benefit in patients, and this difference is statistically significant with p<0.05. Indeed, based on best response, clinical benefit was obtained by only two patients in the low AUC group (irSD>18 weeks), while there are 7 patients in the high AUC group (2 irPR, and 5 irSD>18 weeks). This clearly demonstrated that patients in the high AUC group were signifigantly more likely to obtain clinical benefit.

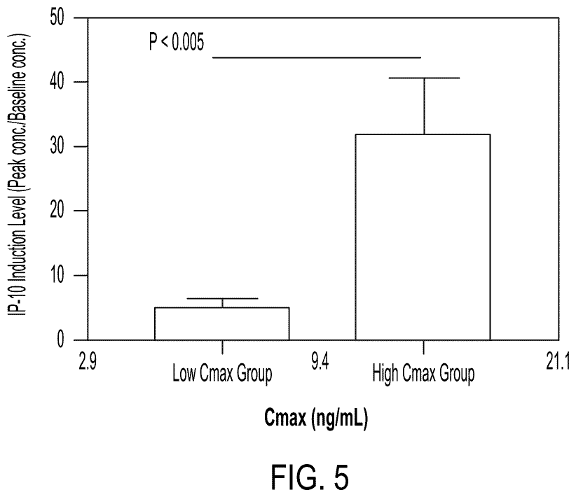

As shown in FIG. 5, IP-10 induction level of was presented as the induction from baseline and divided into two groups in accordance with the Cmax of Compound A, with 16 patients in a low Cmax group (Cmax below 9.4 ng/mL) and 16 patients in the a high AUC group (Cmax above 9.4 ng/mL) group. The mean value of induction level of IP-10 is 5.09-fold for the low Cmax group but 31.9-fold for the high Cmax group, indicating that a minimum of Cmax of 9.4 ng/mL is required to induce enough IP-10 that in turn results in clinical benefit in patients, and this difference is statistically significant with p<0.05. Indeed, based on best response, clinical benefit was obtained by only two patients in the low Cmax group (irSD>18 weeks), while there are 7 patients in the high Cmax group (2 irPR, and 5 irSD>18 weeks). This clearly demonstrated that patients in the high AUC group were signifigantly more likely to obtain clinical benefit.

There was a correlation between higher IP-10 induction and efficacy, more so than IFN-γ. Median IP-10 induction (peak concentration between C1D1 and C1D8) relative to baseline for subjects with clinical beneift (irPR+irSD>18 weeks) was 13.5-fold compared to 4.0-fold and 3.4-fold in subjects with irSD<18 weeks and uirPD, respectively. Likewise, median IFN-γ induction relative to baseline for subjects with clinical beneifts (irPR+irSD>18 weeks) was 7.5-fold compared to 3.0-fold and 2.5-fold in subjects with irSD<18 weeks and uirPD, respectively. It is clear that higher dose levels produce greater IP-10 or IFN-γ induction which in turn correlates to better clinical efficacy.

### Example 6

### Compound A Sulfate Salt Injection Solution

A dosage form was manufactured to provide 1.5 mL of solution of Compound A sulfate at a concentration of 1.31 mg/ml (1 mg free base per mL) in a 2 mL injection vial. The master formula for making the solution was (per 1 mL):

| | |
|---|---|
| Compound A sulfate | 1.31 mg |
| Sodium chloride | 9.1 mg |
| Sodium citrate dihydrate | 0.71 mg |

Citrate dehydrate or Citric acid to adjust pH to 4.0-5.0
Water for injection to adjust volume to 1 mL

Compound A sulfate is C₁₇H₂₂N₄O₂·H₂SO₄, molecular weight 412.46. The crystal form was confirmed during the production (crystal form A, as identified in WO2018232725, which is hereby incorporated by reference).

To administer, an appropriate volume is withdrawn from the injection vial and injected into an intravenous solution container (for example, 100 mL of normal saline), which is infused intravenously into the patient. For example, for a patient with a BSA of 1.80 who is to receive a dose of 0.75 mg/m² of Compound A free base, that is, 1.35 mg, a volume of 1.35 mL of the Compound A sulfate solution would be withdrawn from the injection vial and injected into the intravenous solution container.

### Example 7

### Correlation of Compound A in Monotherapy or in Combination with Anti-PD-1 or Anti-PD-L1 Therapy and Clinical Efficacy

Compound A has been evaluated in monotherapy and in combination with an anti-PD-1 mAb (pembrolizumab) or an anti-PD-L1 mAb (atezolizumab) in subjects with advanced solid tumors. An analysis was performed to investigate the correlation between clinical benefit and the pharmacokinetic parameters of the free base form of Compound A when administered in monotherapy or in combination with the anti-PD-1 mAb or anti-PD-L1 mAbs. Clinical benefit was defined as radiographic complete response, partial response or stable disease >18 weeks based on Response Evaluation Criteria in Solid Tumors (RECIST) version 1.1.

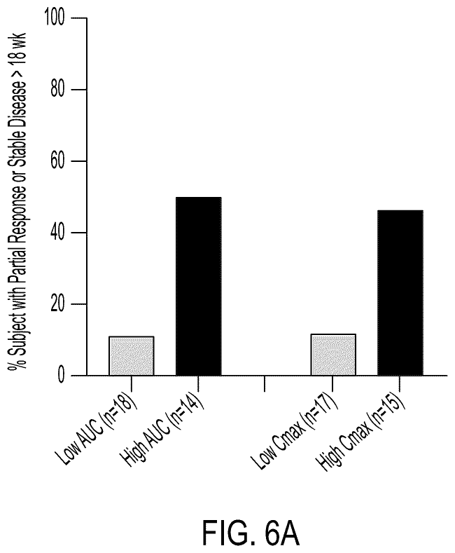

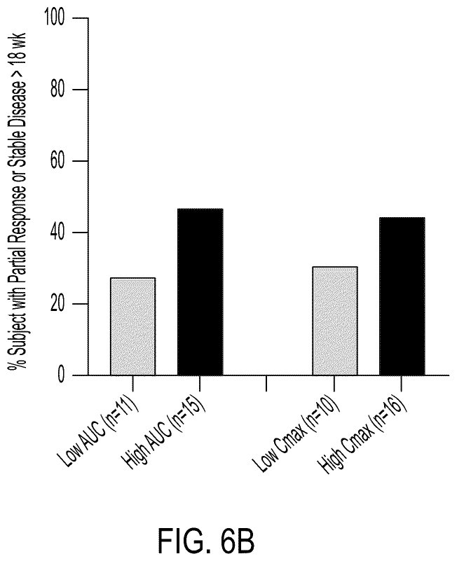

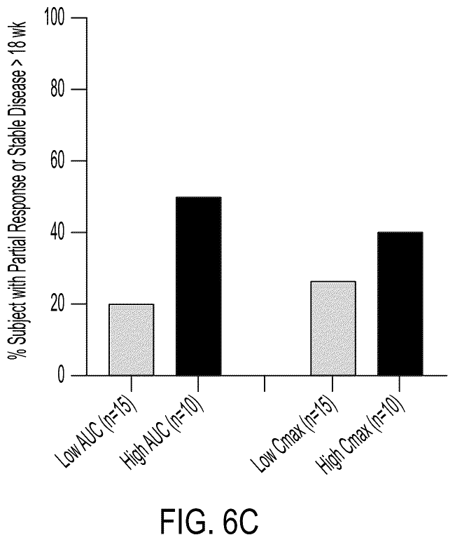

As shown in FIG. 6A, Compound A was evaluated in monotherapy in 32 patients with advanced solid cancers. The patients divided into two groups in accordance with the AUC for Compound A, with 18 patients in a low AUC group (AUC below 3.42 ng/mL*Day) and 14 patients in the high AUC group (AUC above 3.42 ng/mL*Day). Clinical benefit to the treatment was observed in 11% of patients in the low AUC group compared to 50% of patients in the high AUC group, indicating that a minimum of AUC 3.42 ng/mL*Day is more likely to produce clinical benefit in patients. Evaluation of Cmax in the same patients divided into two groups in accordance with the Cmax for Compound A, with 17 patients in a low Cmax group (Cmax below 8.38 ng/mL) and 15 patients in the high Cmax group (Cmax above 8.38 ng/mL). Clinical benefit to the treatment was observed in 12% of patients in the low Cmax group, compared to 47% of patients in the high Cmax group, indicating that a minimum of Cmax 8.38 ng/mL is more likely to produce clinical benefit in patients.

As shown in FIG. 6B, Compound A was evaluated in combination with pembrolizumab in 26 patients with advanced solid cancers. The patients divided into two groups in accordance with the AUC for Compound A, with 11 patients in a low AUC group (AUC below 2.19 ng/mL*Day) and 15 patients in the high AUC group (AUC above 2.19 ng/mL*Day). Clinical benefit to the treatment was observed in 27% of patients in the low AUC group compared to 47% of patients in the high AUC group, indicating that a minimum of AUC 2.19 ng/mL*Day is more likely to produce clinical benefit in patients. Evaluation of Cmax in the same patients divided into two groups in accordance with the Cmax for Compound A, with 10 patients in a low Cmax group (Cmax below 7.65 ng/mL) and 16 patients in the high Cmax group (Cmax above 7.65 ng/mL). Clinical benefit to the treatment was observed in 30% of patients in the low Cmax group, compared to 44% of patients in the high Cmax group, indicating that a minimum of Cmax 7.65 ng/mL is more likely to produce clinical benefit in patients.

As shown in FIG. 6C, Compound A was evaluated in combination with atezolizumab in 25 patients with advanced solid cancers. The patients divided into two groups in accordance with the AUC for Compound A, with 15 patients in a low AUC group (AUC below 2.14 ng/mL*Day) and 10 patients in the high AUC group (AUC above 2.14 ng/mL*Day). Clinical benefit to the treatment was observed in 20% of patients in the low AUC group compared to 50% of patients in the high AUC group, indicating that a minimum of AUC 2.14 ng/mL*Day is more likely to produce clinical benefit in patients. Evaluation of Cmax in the same patients divided into two groups in accordance with the Cmax for Compound A, with 15 patients in a low Cmax group (Cmax below 10.5 ng/mL) and 10 patients in the high Cmax group (Cmax above 10.5 ng/mL). Clinical benefit to the treatment was observed in 27% of patients in the low Cmax group, compared to 40% of patients in the high Cmax group, indicating that a minimum of Cmax 10.5 ng/mL is more likely to produce clinical benefit in patients.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating cancer in a patient in need thereof comprising administering 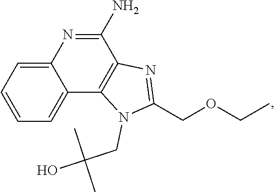 [SMILES: CCOCc1nc2c(N)nc3ccccc3c2n1CC(C)(C)O]
or a pharmaceutically acceptable salt thereof, (Compound A), as a monotherapy, at a dosage of from 0.10 mg/m² to 1.2 mg/m² of the free base.

2. The method of claim 1, wherein the dosage of Compound A is from 0.30 mg/m² to 0.75 mg/m².

3. The method of claim 1, wherein the dosage of Compound A is about 0.75 mg/m².

4. The method according to any one of claims 1-3, wherein the patient has been previously treated with immune checkpoint inhibition therapy.

5. The method of claim 4, wherein the patient's cancer progressed during the previous treatment.

6. The method according to claim 4 or 5, wherein Compound A monotherapy is initiated 2 weeks to 6 months after the last administration of the previous treatment.

7. The method of claim 6, wherein Compound A monotherapy is initiated within 12 weeks after the last administration of the previous treatment.

8. The method according to any one of claims 4-7, wherein the previous treatment further comprised chemotherapy.

9. The method of claim 8, wherein the chemotherapy was cytotoxic therapy.

10. The method of claim 8, wherein the chemotherapy was targeted therapy.

11. The method according to any one of claims 4-10, wherein the immune checkpoint inhibitor therapy was PD-1/PD-L1 blockade.

12. The method of claim 11, wherein PD-1/PD-L1 blockade comprised administration of an anti-PD-1 antibody.

13. The method of claim 11, wherein PD-1/PD-L1 blockade comprised administration of an anti-PD-L1 antibody.

14. The method according to any one of claims 1-13, further comprising quantification of interferon inducible protein 10 (IP-10) concentration or transcript levels in plasma or whole blood before a first dose of Compound A to establish a baseline level and after 6 doses of Compound A to determine a response level.

15. The method of claim 14, wherein if the plasma or whole blood concentration of IP-10 after 6 doses of Compound A is not at least 2-fold greater than the baseline level of IP-10, the dosage of Compound A is increased if the initial dosage of Compound A was less than 0.90 mg/m².

16. A method of treating cancer in a patient in need thereof, comprising administering a therapeutically effective amount of Compound A to the patient to provide a plasma concentration profile in the patient comprising:
a. a maximum plasma concentration (Cₘₐₓ) of Compound A free base of more than 8 ng/mL; and/or
b. an area under the curve (AUC) of Compound A free base of more than 3 ng/mL*Day.

17. A method of treating cancer in a patient in need thereof, comprising administering a therapeutically effective amount of Compound A and an anti-PD-1 antibody to the patient to provide a plasma concentration profile in the patient comprising:
a. a maximum plasma concentration (Cₘₐₓ) of Compound A free base of more than 7 ng/mL; and/or
b. an area under the curve (AUC) of Compound A free base of more than 2 ng/mL*Day.

18. A method of treating cancer in a patient in need thereof, comprising administering a therapeutically effective amount of Compound A and an anti-PD-L1 antibody to the patient to provide a plasma concentration profile in the patient comprising:
a. a maximum plasma concentration (Cₘₐₓ) of Compound A free base of more than 10 ng/mL; and/or
b. an area under the curve (AUC) of Compound A free base of more than 2 ng/mL*Day.

19. The method according to any one of claims 16-18, wherein a mean time to Cₘₐₓ (Tₘₐₓ) is from 15-90 minutes after the administration.

20. The method according to any one of claims 16-18, wherein the Compound A is in a dosage form administered systemically.

21. The method according to claim 20, wherein the dosage form is administered via a parenteral injection route comprising intravenous, intraperitoneal, intramuscular, subcutaneous, or intradermal.

22. The method according to claim 21, wherein the intravenous route comprises intravenous bolus or intravenous infusion.

23. A method of treating cancer in a patient in need thereof comprising administering at least 1-3 doses of an immune checkpoint inhibitor and thereafter administering 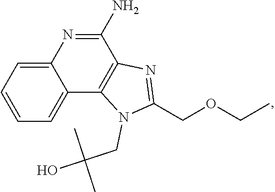 [SMILES: CCOCc1nc2c(N)nc3ccccc3c2n1CC(C)(C)O]
or a pharmaceutically acceptable salt thereof, (Compound A) at a dosage of from 0.10 mg/m² to 1.2 mg/m².

24. The method of claim 23, wherein no further doses of the immune checkpoint inhibitor are administered after the first administration of Compound A.

25. The method of claim 23, wherein treatment with the immune checkpoint inhibitor continues concurrently with treatment with Compound A.
